(19) United States
(12) Patent Application Publication
(10) Pub. No.: US 2008/0300612 A1
(43) Pub. Date: Dec. 4, 2008

(54) Cutting blade storage apparatus

(76) Inventors: Erol D. Riza, Rossford, OH (US); Cosme Ribe Gaya, Woodville, OH (US); James L. Krumm, Toledo, OH (US)

Correspondence Address: EMCH, SCHAFFER, SCHAUB & PORCELLO CO, P O BOX 916, ONE SEAGATE SUITE 1980, TOLEDO, OH, 43697, US

(21) Appl. No.: 12/154,066

(22) Filed: May 20, 2008

Related U.S. Application Data
(60) Provisional application No. 60/931,052, filed on May 21, 2007.

(51) Int. Cl. A61B 17/3215; A61B 19/02; B23P 11/00

(52) U.S. Cl. 606/167; 206/363; 206/350; 29/428

(57) ABSTRACT

A scalpel blade storage device, including a cover having a first lever arm and a second lever arm defined therein and further including a base. The base has a face defining a blade pocket therein having a distal portion adapted to receive a blade portion of a surgical scalpel blade and a proximal portion adapted to receive a shank portion of a surgical scalpel blade. The base is disposed adjacent to the cover, with the face in which the blade pocket is defined facing said cover. A plurality of arms is fixed to one of the base and the cover. The arms envelop at least a portion of the other of the base and the cover to retain the base and the cover in a sliding relationship. The cover is movable between a first closed position in which the blade pocket is completely covered by the cover, and a second open position in which the blade pocket is at least partially uncovered. The invention further includes methods of withdrawing a scalpel blade to the storage device and mounting it on a scalpel handle, and for demounting the scalpel blade and returning it to the storage.

### CROSS-REFERENCE TO RELATED APPLICATIONS

This application claims the priority of provisional patent application No. 60/931,052 filed May 21, 2007.

### BACKGROUND OF THE INVENTION

This invention relates in general to the art of scalpel blade storage, dispensing and disposal, and, in particular, to dispensing and disposal of disposable scalpel blades which can be mounted on blade handles by means of a mating elongated slot and boss. More particularly the invention relates to a device for dispensing and loading a disposable blade onto a handle, and to remove the disposable blade from the scalpel handle after use, and safely containing the used blade for disposal or re-use.

It is known in the art of cutting to provide disposable blades, such as surgical scalpel blades, commercially in several sizes, in sterile or non-sterile packaging, which are adapted to fit conventional metal scalpel handles of various sizes to form knives used for a variety of purposes in hospitals, in research laboratories, and in science departments in schools and universities. Relative to the use in hospitals, such blades are used in surgery, pathology laboratories, etc.

Referring now to FIGS. 11 and 12, commercially available scalpel blades 18 typically have a distal sharpened tip and cutting edge portion 18a and a shank portion 18b extending rearwardly (proximally) therefrom. The shank portion 18b of the surgical blade 18 is provided with an elongated aperture 18c. The elongated handle-engaging aperture of the scalpel blade is preferably provided with a widened proximal (rear) portion 18d and a narrowed distal (forward) portion 18e. Typically, the scalpel blade 18 will have a proximal edge 18f that is inclined to the longitudinal axis of the scalpel blade 18f, to help easily orient the tip and cutting edge portion 18a relative to the associated scalpel handle 19, as will be discussed below. A variety of styles of scalpel blades 18 are available so that a blade appropriate to the task at hand can be used; as used herein, style means both size and shape of the scalpel blade.

The aperture 18c is shaped and adapted to receive a mating fitment in the form of an elongated engaging boss 19a formed on the distal or attaching tip 19b of a scalpel handle 19. The scalpel handle 19 is further provided with a proximal grip portion 19c. A shoulder 19d is preferably formed between the proximal grip portion 19c and the attaching tip 19b of the scalpel handle 19, the shoulder 19d being inclined to the longitudinal axis of the scalpel handle 19, as will be discussed below. The engaging boss 19a is formed with an undercut to engage and guide the scalpel blade 18 on the engaging boss. Suitably, the undercut may be in the form of a pair (only one of which is seen in FIG. 12) of longitudinally extending grooves 19e formed in the lateral walls of the engaging boss 19a.

In assembling the scalpel blade 18 onto the scalpel handle 19, the scalpel handle 19 is initially oriented so that the shoulder 19d is oriented parallel to the proximal edge 18f of the scalpel blade 18. By glancing at the angle of the shoulder 19d, the user will be able to identify which side of the scalpel blade 18 has the sharpened edge, further facilitating use. The widened proximal portion 18d of the aperture 18c initially receives the engaging boss 19a of the scalpel handle 19 to guide the engaging boss 19a forward into the narrowed distal portion 18e of the aperture 18c, as the scalpel blade 18 and the scalpel handle 19 are advanced relatively toward each other. The grooves 19e on either side of the engaging boss 19a receive respective edges of the narrowed distal portion 18e of the aperture 18c with a sliding fit. The scalpel blade 18 bends to accommodate the insertion of the rigid engaging boss 19a into the aperture 18c. When the engaging boss 19a is completely inserted within the blade aperture 18c, the rear edge of the proximal portion 18d of the aperture 18c can be snapped over the rear (proximal end) of the engaging boss 19a (as the scalpel blade 18 straightens out), thus achieving locking engagement between the scalpel blade 18 and the scalpel handle 19.

In order to remove the scalpel blade 18 from the scalpel handle 19, the shank portion 18b of the scalpel blade 18 must be bent outwardly from the scalpel handle 19 to disengage allow the rear edge of the proximal portion 18d of the aperture 18c to clear the proximal end of the engaging boss. The scalpel blade 18 and the scalpel handle 19 can then be moved relatively apart, so that the scalpel blade 18 is pushed forward until the engaging boss 19a disengages narrowed distal portion 18e of the blade aperture 18c, permitting the scalpel blade 18 to straighten out and to be cleared of the scalpel handle 19.

A sharp edge is extremely desirable in conducting a surgical operation. However, blades tend to lose their edge very quickly in such procedures so it is common to use several blades during a single surgical procedure. Thus, removal of a used blade from the scalpel handle and replacement of the scalpel blade with a new sterile blade is a frequent occurrence in the course of a surgical procedure.

The construction and operation of mounting and dismounting scalpel blades on blade handles present problems in that the handling of such conventional surgical blades for mounting and removal purposes can easily cause injury to the handler. Accordingly, it would be desirable to provide a means for safely mounting and unmounting scalpel blades from scalpel handles.

### SUMMARY OF THE INVENTION

This invention relates to a scalpel blade storage device, and methods for use thereof. The device includes a cover having a first lever arm and a second lever arm defined therein and further includes a base. The base has a face defining a blade pocket therein having a distal portion adapted to receive a blade portion of a surgical scalpel blade and a proximal portion adapted to receive a shank portion of a surgical scalpel blade. The base is disposed adjacent to the cover, with the face in which the blade pocket is defined facing said cover. A plurality of arms is fixed to one of the base and the cover. The arms envelop at least a portion of the other of the base and the cover to retain the base and the cover in a sliding relationship. The cover is movable between a first closed position in which the blade pocket is completely covered by the cover, and a second open position in which the blade pocket is at least partially uncovered.

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1 shows a perspective view of an embodiment of a scalpel blade storage device according to the invention, with a cover of the device in a closed position relative to a base of the device.

FIG. 2 shows a perspective view of the device, with a cover portion of the device in an open position relative to the base of the device.

FIG. 3 shows an exploded view of the device, with the cover removed from the base, the base having a disposable scalpel blade disposed in a pocket formed in the base.

FIG. 4A shows a bottom plan view of the device in the closed position.

FIG. 4B shows a side elevation view of the device in the closed position.

FIG. 5 shows a bottom perspective view of the cover of the device.

FIG. 6 shows a perspective view of the device, together with a handle for the disposable scalpel blade.

FIG. 7 is a cross-sectional view of the device, disposable cutting scalpel blade, and handle taken along the line 7-7 of FIG. 6, illustrating the beginning of the process for loading the scalpel blade onto the scalpel handle.

FIG. 8 is a cross-sectional view of the device, disposable scalpel blade, and handle taken along the line 7-7 of FIG. 6, illustrating the scalpel blade mounted onto the scalpel handle.

FIG. 9 is a cross-sectional view of the device, disposable scalpel blade, and handle taken along the line 7-7 of FIG. 6, illustrating the beginning of the process for unloading the scalpel blade from the scalpel handle.

FIG. 10A is a view similar to FIG. 9, except showing a later step in the process of unloading, where the device is in a closed position and the scalpel blade is bent to permit unmounting of the scalpel blade from the scalpel handle.

FIG. 10B is a cross-sectional view of the device, scalpel blade, and scalpel handle taken along the line 10B-10B of FIG. 10A.

FIG. 10C is a cross-sectional view of the device, scalpel blade, and scalpel handle taken along the line 10C-10C of FIG. 10A.

FIG. 10D is a cross-sectional view of the device, scalpel blade, and scalpel handle taken along the line 10D-10D of FIG. 10A.

FIG. 11 is a perspective view of a typical scalpel blade of the prior art.

FIG. 12 is a perspective view of a typical scalpel handle of the prior art.

FIG. 13 is a flow chart illustrating a method of removing a scalpel blade from a storage device and mounting the scalpel blade on a scalpel handle, according to the invention.

FIG. 14 is a flow chart illustrating a method of removing a scalpel blade from a scalpel blade and capturing the scalpel blade in a storage device, according to the invention.

FIG. 15 is a flow chart illustrating a method of manufacturing a storage device according to the invention.

FIG. 16 is a perspective view of the base of the device.

FIG. 17 is a perspective view of the cover of the device.

FIG. 18 is a cross sectional side elevation view of the device with the cover being positioned on the base.

FIG. 19 is a cross sectional side elevation view of the device between the open and closed position.

FIG. 20 is a cross sectional side elevation view of the device in the closed position.

FIG. 21 is a cross sectional side elevation view of the device in the closed position, showing the operation of the locking mechanism.

### DETAILED DESCRIPTION OF THE PREFERRED EMBODIMENT(S)

The following description is of an embodiment of the blade loading, unloading, storage, and disposal device of my invention. Some alternate embodiments are also described. However, there should be no assumption that any particular features or combination of features are required to define the invention, just because such feature(s) appear(s) in one or even all of the figures or discussions of the various embodiments. Rather, this description only serves to describe an embodiment of my invention, along with a few of the many alternate embodiments of my invention; the plain language of the claims should be the only limitation on the scope of the claimed invention. Specifically, no feature described in this specification should be read into the accompanying claims if such feature is not specifically recited in the claim.

Additionally, in the following description of the invention, certain terminology may be used for the purpose of reference only, and are not intended to be limiting on the scope of invention (unless included in claim language). The terms “upper”, “lower”, “above”, “below”, “botom”, “top”, “rightward”, leftward”, “clockwise”, and “counter clockwise” refer to directions in the drawings to which reference is made. The terms “inward” and “outward” refer to directions toward and away from, respectively, the geometric center of the component described. The term “longitudinal” means running lengthwise in a manner generally parallel with a prominent axis of the device (generally the longest axis of the structure under discussion, or, as may be determined by the usage, an axis of motion of the structure). The terms “transverse” means situated or extending across the structure under discussion, substantially perpendicular to the longitudinal axis of the structure under discussion. The term “lateral” means of, at, towards, or from the side or sides of the structure under discussion. The term “proximal” means that portion of the structure under discussion which is normally close to the user when held for use. Similarly, the term “distal” refers to that portion of the structure under discussion which is farther away from the user when held for use. Such terminology used for reference only, and not intended to be limiting on the scope of invention (unless included in claim language) will include the words specifically mentioned above, derivatives thereof, and words of similar import.

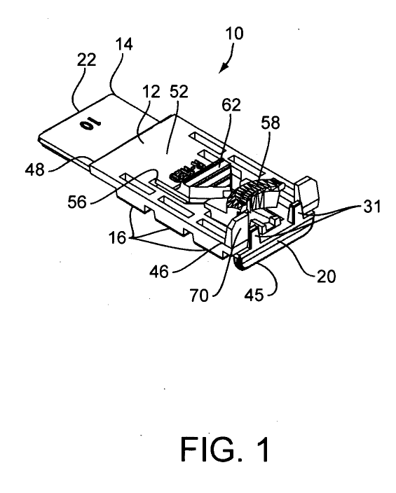

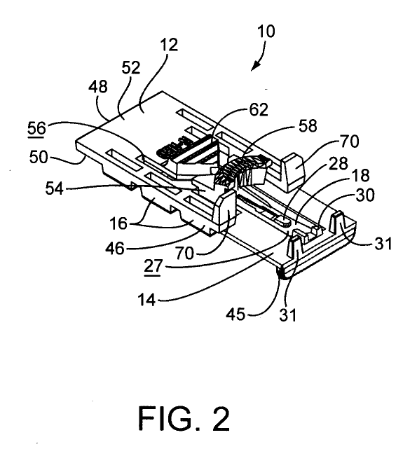

Furthermore, the following discussion refers to a “scalpel blade” and a “scalpel handle”. A preferred embodiment of the device of my invention is used with a surgical scalpel having a scalpel blade which can be separated from the scalpel handle. However, use of the device of my invention is not limited to surgical scalpels—it can be use with any suitable device having a tool tip which can be separated from an associated handle—where the tool tip is provided with an aperture and the handle is provided with a engaging boss (or vice versa) generally similar to that described herein. For example, the device of my invention may be used with a hobby tool, such as hobby knives or carving gouges. FIGS. 1 and 2 are perspective views of one of several embodiments of a device 10 for storing, loading, and/or unloading scalpel blades. As will be seen, the device 10 illustrated in FIGS. 1 and 2 can in fact be used perform the functions of storing an unused scalpel blade, can be operated to load the scalpel blade onto a scalpel handle, unload a scalpel blade from a scalpel handle, and safely store the scalpel blade for disposal or reuse. It is contemplated that some alternate embodiments (not shown) may perform fewer (or more) functions, and, of course, the embodiments capable of performing multiple functions may be used for fewer functions than the device 10 is capable of.

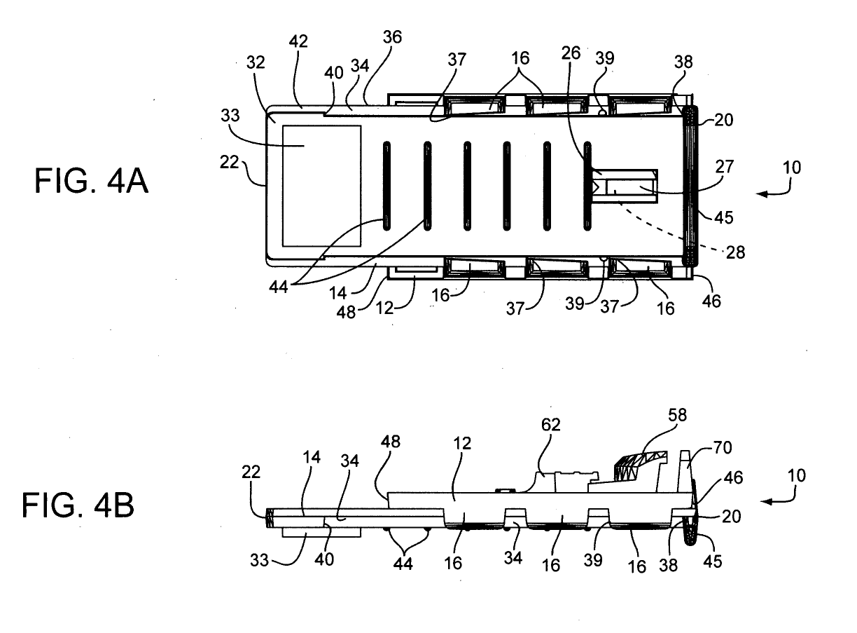

The device 10 includes a cover 12 and a base 14. The cover 12 is provided with a plurality of arms 16, (best seen in FIGS. 4A and 4B) having free ends 37 that at least partially envelop the base 14 to hold the cover and the base together. Note that in alternate embodiments (not shown), one arm 16 per side may be used; or the arms 16 may be formed on the base 14 and partially envelop the cover 12. The cover 12 can slide relative to the base 14. In FIG. 1, the cover 12 is shown in a closed position relative to the base 14. In FIG. 2, the cover 12 is shown in an open position, relative to the base 14, exposing a scalpel blade 18 stored in the base 14. The cover 12 and the base 14 are preferably injection molded of polymeric materials. In a preferred embodiment, the materials are thermoplastic materials, with the base 14 being formed of ABS (Acrylonitrile Butadiene Styrene) and the cover 12 being formed of polypropylene, but the cover 12 and the base 14 may be formed of any material suitable for the desired application (such as thermosetting plastics, other plastic materials, metals, ceramics, composites, or, indeed, any suitable natural or manmade material), and formed by any suitable process (molding, machining, stamping, composite layup, etc.) with the exact arrangement of these components being adjusted to account for the material used, and the manufacturing process used, as is well known in the art. For example, with a molded material it is normally desirable to try to maintain a relatively uniform thickness of material, that is, not have large pockets of material next to thin sections of material, since material may be drawn out of the thin section when the large pocket cools and the cooling plastic material therein contracts. Thus, although not discussed in detail herein, it is to be expected that pockets, holes, draft allowances, etc., would be formed in the molded cover 12 or base 14 to achieve the best result from a molding process, and such features are believed to be well within the ordinary skill of mold makers working in the art, and accordingly need not be discussed in further detail here.

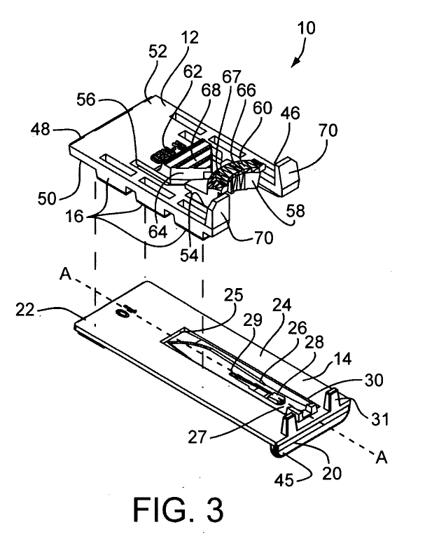

As best seen in FIG. 3, the base 14 is preferably formed as a rectangular plate having a first end 20 and a second end 22. A first face 24 of the base 14 faces the cover 12. A blade pocket 25 is formed in the first face 24. As will be seen, the blade pocket 25 constitutes a blade storage space defined between the cover 12 and the base 14. Suitably, the blade pocket 25 is elongate along an axis “A” extending centrally between the first end 20 and the second end 22 of the base 14, near the first end 20 of the base 14.

An opening 26 is formed through the base 14 in the blade pocket near the first end 20 of the base 14. Preferably, an arm 27 extends into the opening 26 from the wall thereof closest to the first end 20 of the base 14. Also preferably, an upwardly extending (as viewed in FIG. 3) boss 28 (or other projection) is formed on the free end of the arm 27 in the blade pocket 25, which assists in retaining the scalpel blade 18. As will be seen, the boss 28 is received in the distal portion 18e of the elongate aperture 18c formed in the scalpel blade 18. The cooperation there between provides additional assurance that the scalpel blade 18 will not shift laterally during manufacture, shipment and/or storage of the blade device 10, thereby assuring that the tip and cutting edge portion 18a of the scalpel blade 18 will not become dulled or nicked.

A central deeper portion 29 of the blade pocket 25 is formed along the axis “A” distally of the arm 27, which is suitably sized and oriented to receive a portion of the attaching tip 19b of the scalpel handle 19 when mounting and dismounting the scalpel blade 18 from the scalpel handle 19, as will be described below. Thus, the shape and, to some extent, the depth of the blade pocket 25 are related to the shape and thickness of the scalpel blade 18 to be stored therein, as well as the shape of the scalpel handle 19 on which the scalpel blade 18 is or is to be mounted.

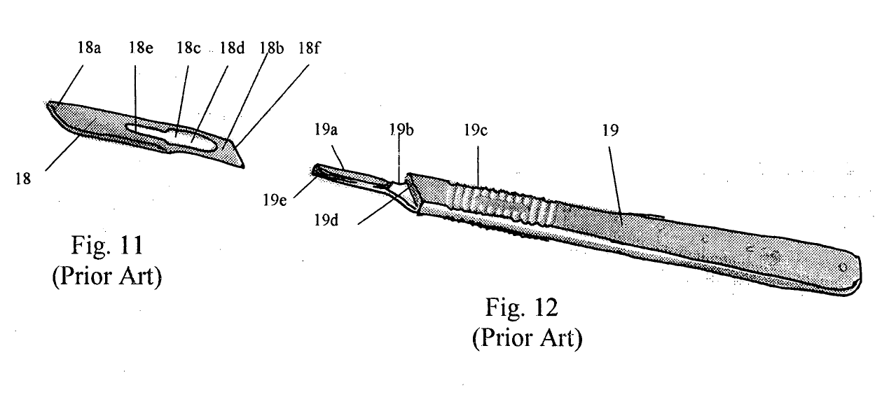

Preferably, a first plurality of positioning bosses 30 are formed on the first face 24 between the blade pocket 25 and the first end 20 of the base 14. The number, shape, and exact position of the positioning bosses 30 will be suitably determined by the shape of the scalpel blade 18 to the retained in the blade pocket 25. The transverse distance between the positioning bosses 30 is based on the transverse width of the attachment tip 19b, so as to guide the scalpel handle into the proper position, transversely, relative to the base 14. As discussed above, the scalpel blade 18 will have a proximal edge 18f that is inclined to the longitudinal axis of the scalpel blade 18, to help easily orient the scalpel blade 18 to the associated scalpel handle 19 (seen in FIGS. 6 and 11). Preferably, therefore, the positioning bosses 30 will be inclined at a similar angle to the axis “A”, so as to abut the proximal end of the scalpel blade 18 when the scalpel blade 18 is disposed in the blade pocket 25. Preferably, one or more alignment bosses 31 are also provided, and more preferably a pair of alignment bosses 31 are formed on either side of the axis “A”. The alignment bosses 31 extend from the first face 24, adjacent the first end 20. The transverse distance between the alignment bosses 31 is based on the transverse width of the grip portion 19c of the scalpel handle 19, so as to guide the scalpel handle 19 into the proper position, transversely, relative to the base 14.

FIG. 4A is a view of a second face 32 of the base 14. Preferably, a body 33 is fixed to the base 14, suitably near the second end 22, as seen in FIGS. 4A and 4B. The body 33 is preferably a magnet, allowing the device 10 to be magnetically attached to a surgical tray, for example, to facilitate keeping order in the surgical suite, and keeping the device 10 conveniently at hand. It will be recognized that the body 33 may be attached almost anywhere on the device 10, and need not be attached in the location shown in FIGS. 4A and 4B. The body 33 may be attached by any suitable method such as being molded into a suitable location on the device 10. In a preferred embodiment, the body 33 is a piece of “tape magnet”, a magnet with an adhesive surface normally dispensed on a roll and cut to size. Also, preferably, the body 33 is formed of a radio-opaque material, such that it will show up on an x-ray, allowing the device 10 to be easily located if it is accidentally dropped into the site of a surgical procedure.

As may be best seen with reference to FIGS. 3, 4A and 4B, the second face 32 has a pair of elongate recesses (rabbets) 34 formed at each edge 36 (only one of which is shown). The recesses 34 receive the free ends 37 of the arms 16 of the cover 12. The recesses 34 each have an end 38 nearest the first end 20 of the base 14. Spaced apart from the ends 38 the width of one of the arms 16 is a slight protrusion 39 into each, preferably, of the recesses 34, the purpose of which will be explained below. The recesses 34 also each have a reduction of width at a shoulder 40 near the second end 22 of the base 14. The ends 38 and the shoulders 40 will each engage the arms 16 of the cover 12, thus blocking movement of the arms 16 engaged in the recesses 34, preventing the ordinary relative movement of the cover 12 and base 14 to beyond the open and closed positions illustrated in FIGS. 2 and 1, respectively. Preferably, a reduced width portion 42 of each recess 34 extends between the respective shoulder 40 and the second end 22 of the base 14, the purpose of which will be explained below. The second face 32 of the base 14 will preferably be provided with a textured surface 44, such as the illustrated molded in series of spaced apart transverse ridges or any other suitable pattern, in order to provide the user with a slip-resistant gripping surface. The second face 32 of the base 14 is also preferably provided with a flange 45 extending perpendicular to the second face 32 along the first end 20 of the base 14. The flange 45 has several purposes, including: Increasing transverse rigidity of the base 14; preventing a users fingers from slipping off the first end 20 of the base 14 when inserting a blade 18 into the device (the flange 45 is preferably of greater height than the textured surface 44); and acting as a minimal shield (to some extent, depending on the height of the flange 45) against accidental cuts of the user's fingers used to hold the base 14 when inserting a scalpel blade 18 into the device 10.

Preferably, either one or both of the first face 24 or the second face 32 of the base 14 is provided with indicia identifying the type (e.g., style and size) of blade 18 that can be accommodated in the blade pocket 25. Such indicia is preferably formed during the molding process, but may be printed on the base or attached thereto after the base is formed. The indicia may be, for example, raised molded, indented molded or printed letters, shapes, or numbers. The indicia may also be a color or combination of colors of the material used to make the base (for example, blue molded plastic base for one a base 14 holding one type of blade 18, red molded plastic for a base 14 holding another type of blade 18, or white and gray mottled plastic for a base 14 holding another type of blade 18). In a preferred embodiment, red plastic is used to make the base 14 for a #11 blade, blue plastic is used to make the base for a #10 blade, and green plastic is used to make the based for a #15 blade. All of these standard blades will fit a scalpel handle 19 with a standard #3 fitment (attachment tip 19b), and a single size of cover 12 can be used with these bases 14. There are another range of scalpel blades 18 types that will fit on a standard #4 fitment (which is larger than the #3 fitment). An associated set of uniquely color-coded bases 14 can be used to hold these scalpel blades 18, and accommodate the larger fitment. To accommodate the larger size fitment, preferably a different size cover 12 will be provided for this set of color-coded bases. Similarly, still different bases 14 and an associated size of cover 12 will be used with scalpel handles 19 that have a fitment, which is even larger than the #4 fitment. The covers 12 may be provided with indicia of the size of the cover 12, that is, the fitment which may be used with that cover 12. The indicia, like those of the base 14, may be any suitable indicia, including color coding.

Referring now to FIGS. 1, 2, 3, and 5, the cover 12 has a first end 46, a second end 48, a first face 50 and a second face 52. The first end 46 is closest to the first end 20 of the base 14, and the second end 48 is closest to the second end 22 of the base 14 when the cover 12 is assembled on the base 14. The plurality of arms 16 extend out of the first face 50, preferably perpendicularly. The free ends 37 of the arms 16 extend inwardly, preferably perpendicularly, relative to the portion of the arms fixed extending perpendicularly from the cover 12, to engage the recesses 34 in the base 14. Thus the arms 16 act to hold the first face 50 of the cover 12 in sliding contact with the first face 24 of the base 14, that is to say, the arms 16 retain the base 14 and the cover 12 in a sliding relationship. The sliding relationship may involve continuous or intermittent contact, depending on how tightly the arms 16 retain the base 14 and the cover 12 together. Preferably, though, the arms 16 retain the base 14 and the cover 12 together sufficiently tightly, or a series of overlapping cooperating flanges or other features (not shown) may be provided on the base 14 and/or the cover 12, that preferably no gaps between the base 14 and the cover 12 are formed which are large enough for the scalpel blade 18 to pass through, other than when the blade pocket 25 is exposed by moving the cover 12 to the open position relative to the base 14.

Referring to FIG. 4A, the free ends 37 of the arms 16 are tapered outwardly from a side closest to the second end 46 to the side closest to the first end 20 of the cover 12. The distance between two transversely opposed arms 16 measured between the respective sides of the free ends 37 closest to the first end 46 is greater than the distance between the transversely opposed reduced width portions 42 of the recesses 34 on the base 14. In contrast, the distance between two transversely opposed arms 16 measured between the respective sides of the free ends 37 closest to the second end 48 is less than the distance between the transversely opposed reduced width portions 42 of the recesses 34 on the base 14, when the cover 12 is in a relaxed condition.

This taper on the free ends 37 facilitates assembly of the cover 12 onto the base 14 by allowing the side of the arms 16 closest to the first end 46 of the cover 12 to freely engage the recesses 34 on the base 14. With the arms 16 thus partially engaging the recesses 34 on the base 14, the cover 12 is pushed axially toward the first end 20 of the base 14, bending the arms 16 and distorting the cover 12 to allow the sides of the free ends 37 of the opposing pairs of arms 16 closest to the second end 48 to pull apart a sufficient distance to slide into the reduced width portion 42 of the recesses 34. Once past the shoulders 40, the arms 16 spring inwardly to engage the full width portion of the recesses 34, allowing the cover 12 to return to an undistorted condition. The reduced width portion 42 thus cooperates with the arms 16 to capture the cover 12 on the base 14 to slide between the open and closed positions illustrated in FIGS. 2 and 1, respectively.

The protrusion 39 near the second end 22 of the base 14 interferes slightly with the innermost tip of the free end 37 of an associated one of the arms 16 such that the arm 16 flexes slightly when the free end 37 goes past the protrusion 39 just before reaching the closed position of FIG. 1. The arm 16 can spring back when the cover 12 is fully in the closed position of FIG. 1. When fully in the closed position, the cover 12 is held in the closed position by the interference of the tips of the free ends 37 of the arm 16s and the protrusions 39. However, the interference is so small that the cover 12 is not prevented from being deliberately moved from the closed position to the open position by a user, with the arms 16 flexing again as they are moved past the protrusions 39.

Three slots are formed through the cover 12 from the first face 50 through to the second face 52. A first pair of slots 54 is transversely spaced apart, and extend into the cover 12 from the first end 46, parallel to the axis “A” of the base 14. A third, generally U- or V-shaped slot 56 is formed through the central portion of the cover 12. Between the first pair of slots 54 is a first lever arm 58, having a free end 60 at the first end 46 of the cover 12. The third slot 56 extends around the free portion of a second lever arm 62, which has a generally centrally disposed free end 64, and which is fixed near the second end 48 of the cover 12. Preferably, the first lever arm 58 has an upwardly (as viewed in FIGS. 1 and 2) extending boss 66 formed on the free end 60. In plan view, the boss 66 preferably generally extends transversely across the end of the first lever arm 58 so as to describe an arc about a point somewhat beyond the first end 46 of the cover 12. The boss 66 also preferably has a flange formed thereon which extends slightly toward the first end 46 of the cover 12, to provide a grip area 67 to be gripped by a user when operating the first lever arm 58. The grip area 67 of the boss 66 may suitably be provided with a texture to provide slip resistant grip for the user. Also, preferably, the second lever arm 62 has an upwardly (as viewed in FIGS. 1 and 2) extending boss 68 formed on the free end 64. The boss 68 may also suitably be provided with a textured grip area 69 to provide slip resistant grip for the user.

Preferably, a plurality of bosses 70 (suitably, a pair) are formed on either side of the first end 46 of the cover 12, extending perpendicularly from the second face 52. When the cover 12 is in the closed position (illustrated in FIG. 1) the bosses 70 on the cover are transversely outboard of a respective one of the alignment bosses 31 on the base, and, like the alignment bosses 31 and the positioning bosses 30, help guide the user when inserting the scalpel handle 19 into the device 10 when the cover 12 is in the open position (illustrated in FIG. 2), and thus helps guard against accidental cuts when the scalpel blade 18 is mounted on the scalpel handle 19.

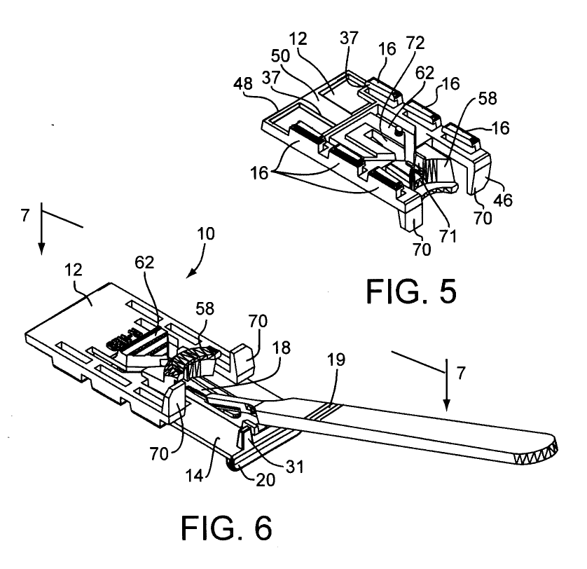

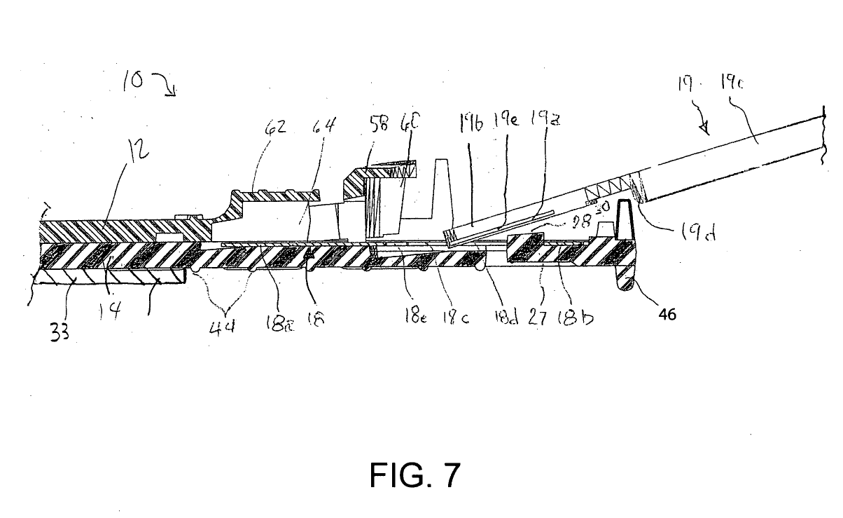

Referring now to FIG. 5, which is a view of the first face 50 of the cover 12, the first lever arm 58 is shown having a longitudinally extending recess 71 formed therein on the face thereof opposite the boss 66, the purpose of which will be described below. Similarly, the second lever arm 62 has a longitudinally extending recess 72 formed therein on the face thereof opposite the boss 68, the purpose of which will be described below.

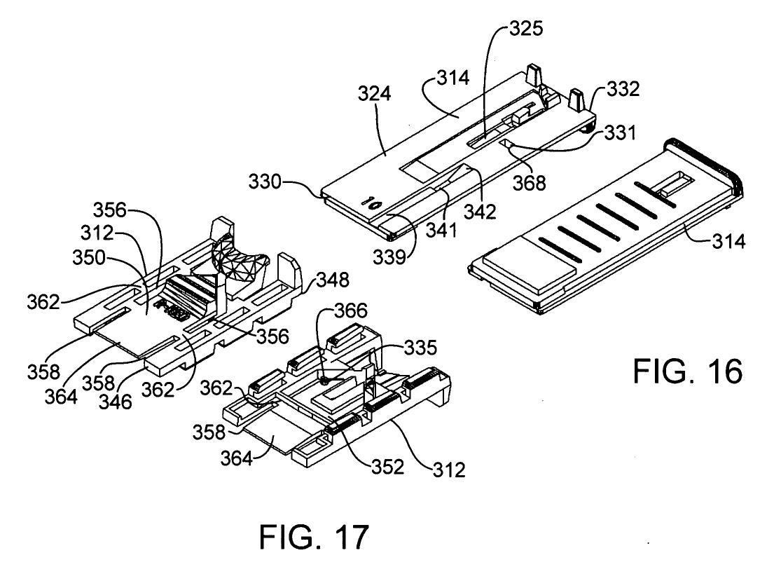

FIGS. 16-21 show an additional feature that can be utilized with the blade loading, unloading, storage and disposal device of the present invention. The device 300 shown in these figures is substantially similar to the device 10 previously described and the device 300 function in substantially the same manner as the previously described device 10. The prior description concerning the structure and operation of the device 10 applies equally to the device 300 and only the additional features that have been added to the device 300 will be described in detail. The device 300 has a base 314 and a cover 312 that is slidably positioned on the base 314 as previously described. The base 314 has a blade pocket 325 to store a cutting blade (not shown) until the cutting blade is ready to be used and is removed from the blade pocket 325 as previously discussed. The base 314 has a first face 324 and the blade pocket 325 is located on the first face of the base 314. A first locking device 331 is positioned on the first face 324 of the base 314. The first locking device 331 is usually positioned on the first face 324 adjacent to the blade pocket 325. However, it should be recognized that other locations on the first face 324 can be utilized for the location of the first locking device 331. Also positioned on the first face 324 of the base 314 is a channel 339. The channel 339 extends from the first end 330 of the base 314 in a direction towards first locking device 331. The channel 339 extends in a direction that is substantially parallel to the longitudinal axis that extends between the first end 330 to the second end 332 of the base 314. The channel terminates at a chamber 341 that is spaced apart from the first end 330 of the base 314. The chamber 341 has a depth when measured from the first face 324 of the base 314 that is greater than the depth of the channel 339. At the end of the chamber 341 that is spaced apart from the channel 339 there is a ramp 343 that extends from the chamber 341 to the first face 324 of the base 314. The ramp 343 can have a slope from about 20° to 45° with respect to the plane defined by the first face 324 of the base 314.

The cover 312 has a first end 346 and a second end 348. The first and second ends of the cover 312 are disposed generally adjacent the first and second ends of the base 314. The cover has a first face 350 and a second face 352. A first pair of slots 356 and a second pair of slots 358 are located in the cover 312 on opposite sides of the cover. The first pair of slots 356 and the second pair of slots 358 are located parallel to the longitudinal axis of the cover 312 and in the first pair of slots 356 and the second pair of slots 358 are in alignment on the cover 312. The first and second pair of slots defines pivot points 362 that define a pivot axis for the portion 360 of the cover 312 that is located between the first pair 356 and second pair 358 of slots. The pivot axis extends between the pivot points 362 and the pivot axis is substantially perpendicular to the longitudinal axis of the cover 312. The portion 364 of the cover 312 that is located between second pair of slots 358 reduces in thickness as the portion extends from the pivot axis defined by the pivot points 362 to the first end 346 of the cover 312. Positioned on the second face 352 of the cover 312 is a second locking device 335. The second locking device 335 is disposed to be in alignment with the channel 339 in the base 314 and with the first locking device 331 when the cover 312 is slidably positioned on the base 314 as previously described. The first locking device 331 and the second locking device 335 can either be a projection 366 or a cavity 368. However, the first locking and the second locking device cannot both be a projection or a cavity. In other words, the first locking device must be a projection 366 if the second locking device is a cavity 368. The projection 366 extends from the base 314 or the cover 312 a distance that is less than the depth of the chamber 341. For the sake of explanation, the first locking device 331 has been shown as a cavity 368 in the drawings. Accordingly, the second locking device 335 is shown as a projection 366 in the drawings.

The portion 360 of the cover 312 that is located between the first pair of slots 356 and the second pair of slots 358 is disposed to be substantially parallel to the first face 350 of the cover. The material of the portion 360 is selected to have a memory that acts to maintain the portion 360 in the orientation. If the portion 360 is displaced from its position that is substantially parallel to the first face 350, the material of the portion 360 will create a biasing force that acts to return the portion 360 back to its original position that is parallel to the first face 350. The biasing force created by the portion 360 can be overcome by the grip force present in the hand of an adult human. However, the biasing force of the portion 360 is sufficient to maintain the portion in its desired position until an external force is applied to the portion 360 to displace the portion.

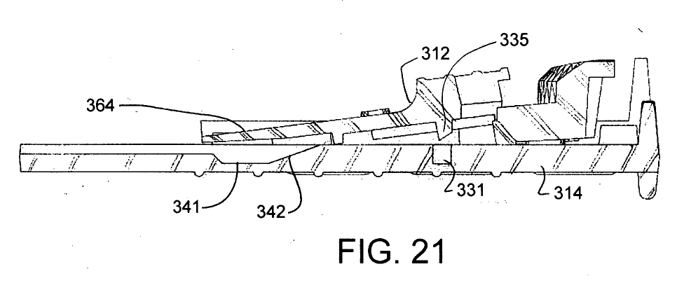

The operation of these additional features will be more readily understood by reference to FIGS. 16-21. FIGS. 20 and 21 show the device 300 with the base 314 and the cover 312 in the closed position. In this position the cover 312 extends over the blade pocket 325 formed in the base 314. In addition, as shown in FIG. 20 the first locking device 331, which is a cavity 368, is in alignment with the second locking device, which is a projection 366, on the cover 312. The first and second locking devices as shown in FIG. 20 are in engagement and the engagement of the first and second locking devices prohibits the sliding movement of the cover 312 with respect to the base 314. As shown in FIG. 20 the cover 312 is locked in position on the base 314 by the engagement of the first and second locking devices. The biasing force produced by the portion 360 of the cover 312 functions to maintain the second locking device 335, which is a projection 366, in engagement with the first locking device 331, which is a cavity 368. The engagement of the first and second locking devices ensures that the cover 312 will be over the cutting blade contained in the blade pocket 325 until it is desired to remove the cutting blade from the blade pocket 325.

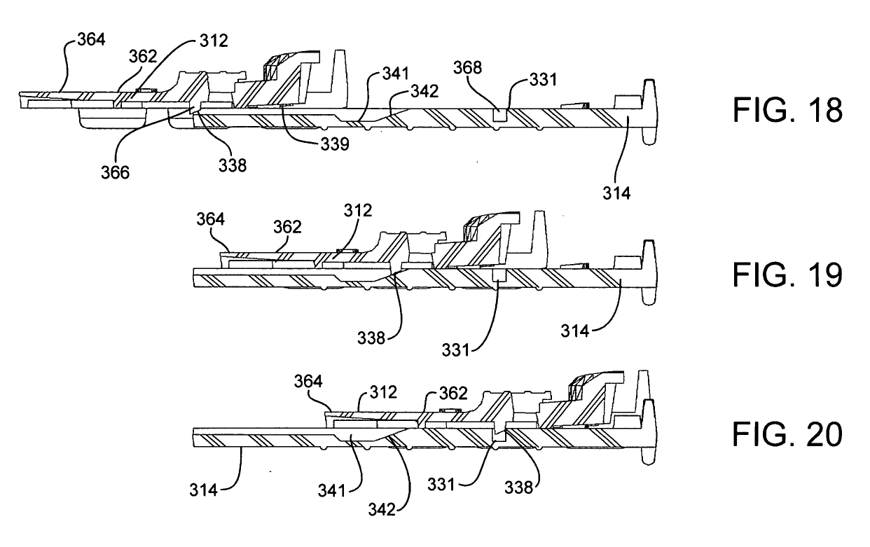

As shown FIG. 21 it is necessary to apply a biasing force to the portion 364 of the cover 312 that is sufficient to overcome the biasing force of the portion 360. When a sufficient external biasing force is applied to the portion 364, the portion 360 will rotate around the pivot axis defined by the pivot points 362 on the cover 312. Rotation of portion 360 of the cover 312 causes the first and second locking devices to disengage so that the cover 312 can be advanced in a direction towards the first end 330 of the base 314 to uncover the blade pocket 325. When the blade pocket is uncovered a cutting blade can be removed from or positioned in the blade pocket 325. The projection 366 on the cover 312 is positioned in the chamber 341 when the blade pocket 325 is uncovered sufficiently to allow for the removal or replacement of the blade from the blade pocket. In this position, the first end 346 of the cover 312 can be rotated around the pivot points 362 towards the blade pocket 325 to allow the first end 346 to engage the blade and hotel the blade in a secure position for the attachment or detachment of a handle for the blade. As the projection is position in the chamber 341 on the base 314, the projection does not prevent this movement for the cover 312. FIG. 18 shows the cover 312 in the second or fully opened position with respect to the base 314. The second locking device 335, which is a projection 366, is positioned in the channel 339 in the base 314 as the cover 312 is advanced to the second or open position. When it is desired to move the cover 312 back to the closed position the second locking device 335, which is projection 366, can be advanced along the channel 339 in the base 314 as the cover 312 is advanced with respect to the base 314. The projection 366 will move along the channel 339 until it encounters the chamber 341. At the end of the chamber 341, the projection 366 will engage the ramp 342 which gradually raises the projection in the portion of the cover 312 that is located between the first pair 356 and second pair 358 of slots. The ramp 342 gradually displaces the projection 366 in a direction away from the channel 339 until the projection 366 is in engagement with the first face 324 of the base 314. In this position the projection has displaced the portion 360 from its normal position and created a biasing or restoring force in the portion 360 that acts toward the base 314. In this position the cover 312 can be advanced towards the second end 332 of the base 314 until the projection 366 comes into alignment with cavity 368 where upon the biasing force of the of the portion 360 of the cover 312 will rotate of the cover 312 so that the projection 366 on the cover 312 is in engagement with the cavity 368 on the base 314. The biasing force of the portion 360 will retain the projection 366 in position in the cavity 368 until a force is applied to the portion 364 to overcome the biasing force of the portion 360 to displace the projection from the cavity. Although, the first locking device has been described as being a cavity in the base 314 and the second locking device as a projection in the cover 312 it should be appreciated that the cavity 368 could be located in the cover 312 and the projection 366 could be located in the base 314 without affecting the functioning of the locking feature for the device 300. In addition, other first and second locking devices could be utilized to maintain the cover 312 in a lock position with respect to the base 314. Of course, the first and second locking devices must be releaseably engageable so that the cover can be moved to an unlocked position whereby the cover can be displaced with respect to the base 314 to move the cover to the open position that allows access to the blade pocket 325.

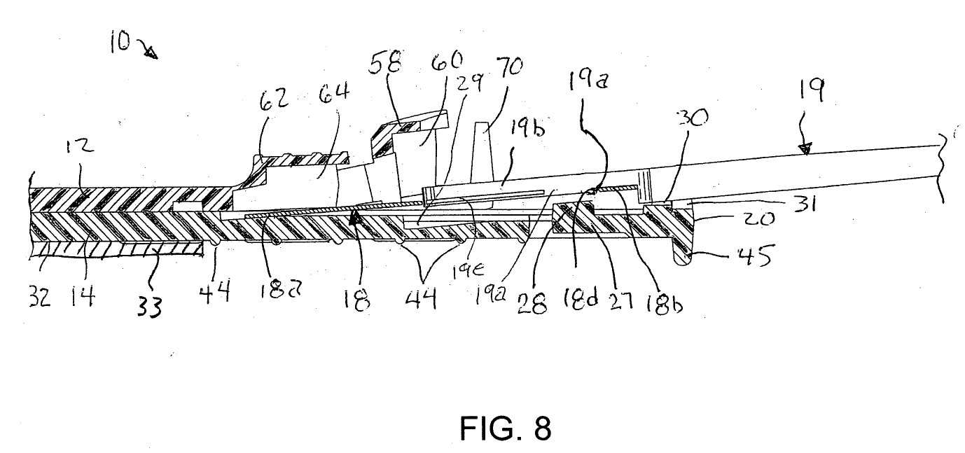

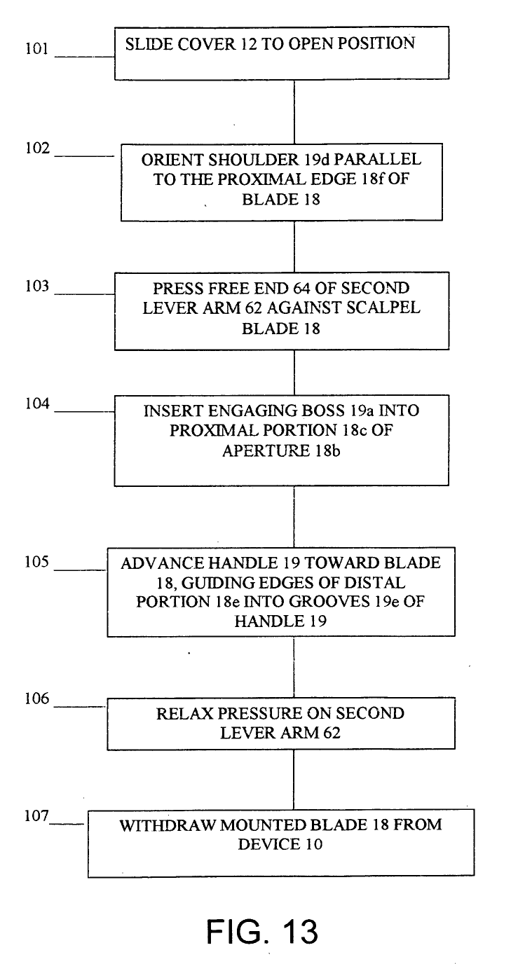

Referring again to FIG. 1, it is contemplated that the device 10 would be supplied in sterile packaging (not shown), holding the device 10 in the illustrated closed position. After disposing of the packaging, in a first step 101 (See FIG. 13) a user will slide the cover 12 to the open position illustrated in FIG. 2, in preparation for loading the scalpel blade 18 onto the conventional scalpel handle 19. In a second step 102, the user orients the shoulder 19d parallel to the proximal edge 18f of the scalpel blade 18. In a third step 103, the user presses the free end 64 of the second lever arm 62 against scalpel blade 18, specifically the tip and cutting edge portion 18a of the scalpel blade 18, as seen in FIGS. 5 and 6. This clamps the tip and cutting edge portion 18a between the second lever arm 62 and the base 14, to stabilize and fix the position of the scalpel blade 18 in the blade pocket 25. In a fourth step 104, the user inserts the engaging boss 19a into the proximal portion 18c of the aperture 18b, as seen in FIG. 6. In a fifth step 105, the user advances the scalpel handle 19 toward the scalpel blade 18; more specifically, the user pushes the attaching tip 19b into the distal portion 18e of the aperture 18c in the scalpel blade 18, guiding the edges of the distal portion 18e into the grooves 19e of handle 19. The scalpel blade 18 bends to accommodate the insertion of the rigid engaging boss 19a into the aperture 18c, and disengages from the boss 28 of the arm 27 in the base 14. When the engaging boss 19a is fully inserted within the blade aperture 18c, the rear edge of the proximal portion 18d of the aperture 18c will snap over the rear (proximal end) of the engaging boss 19a (as the scalpel blade 18 straightens out), thus achieving locking engagement between the scalpel blade 18 and the scalpel handle 19. In a sixth step 106, the user relaxes pressure on the second lever arm 62, freeing the scalpel blade 18, as seen in FIG. 8. In a seventh step, 107, the user withdraws the scalpel blade 18, mounted on the scalpel handle 19 from the device 10.

Once the scalpel blade 18 has been used in a surgical procedure (or for whatever reason it is desired to de-mount the scalpel blade 18 from the scalpel handle 19), the following steps may be employed to re-position the (used) scalpel blade 18 in the original device 10 for storage and/or disposal purposes.

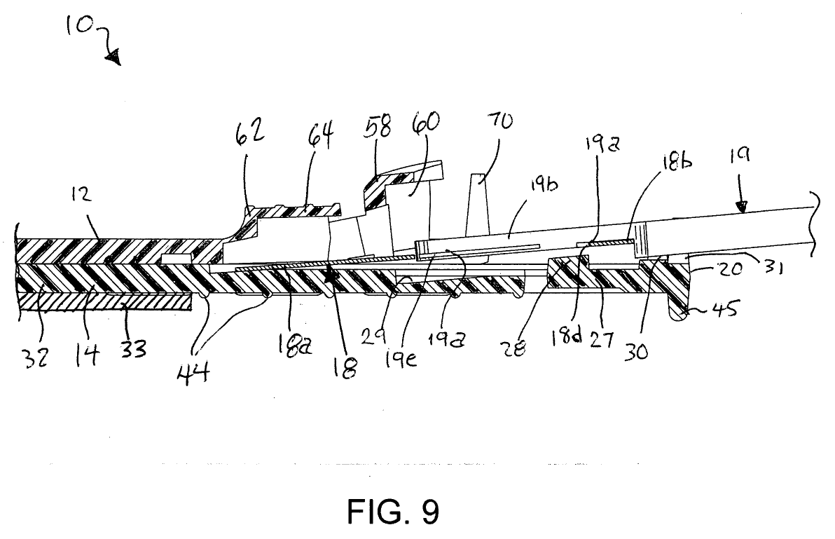

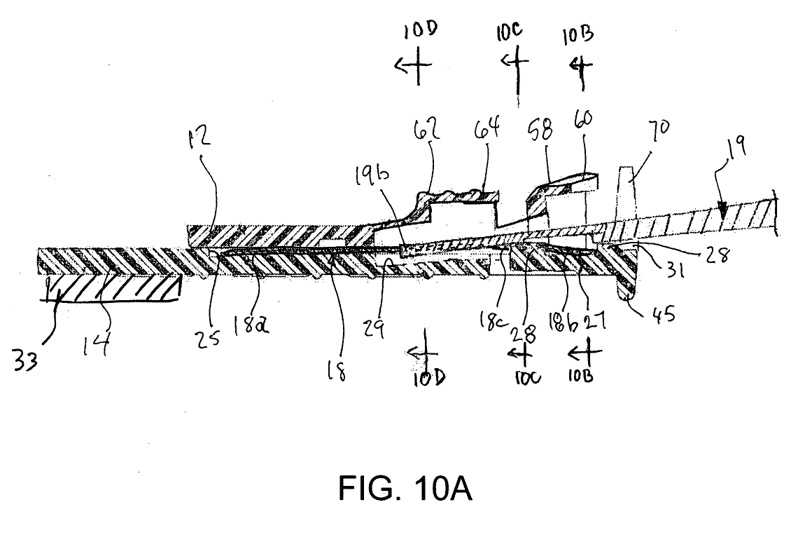

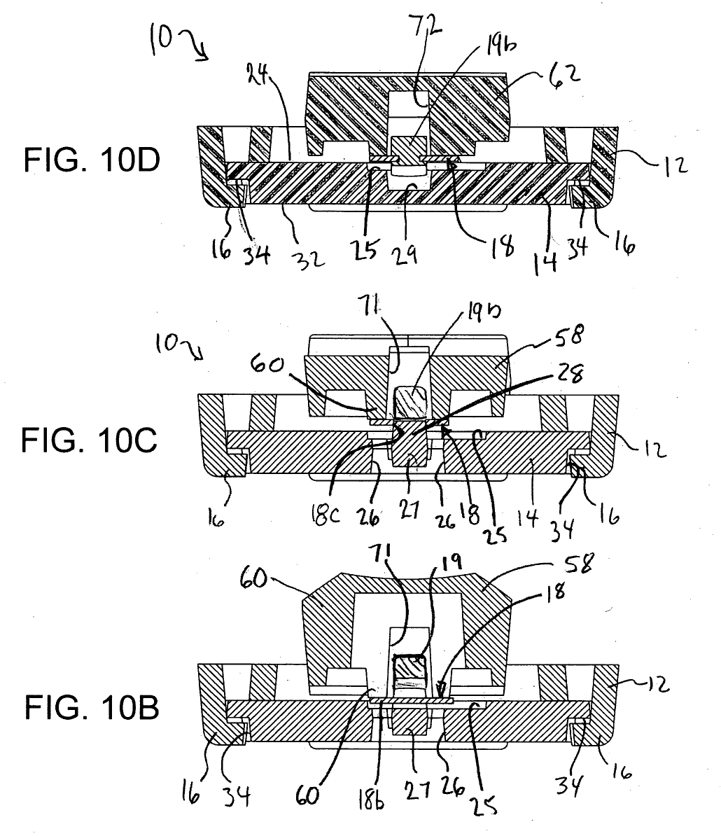

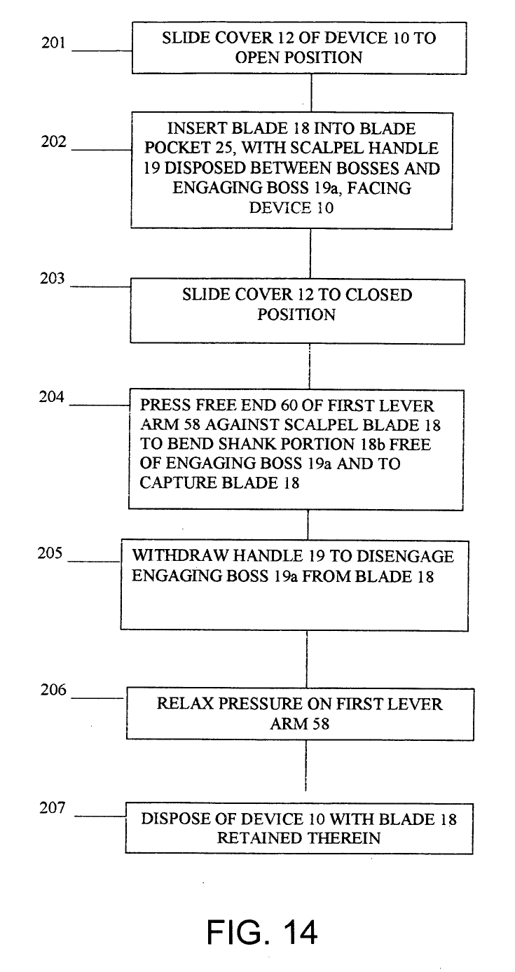

This reverse procedure is shown in FIG. 14. In a first step 201, the user verifies that the cover 12 is in the open position, or moves the cover 12 to the open position illustrated in FIG. 2. Suitably, the scalpel handle 19 can then be held in one hand, and the opened device 10 is held in the other. In a second step 202, the scalpel blade is inserted into the device 10, and more specifically into the blade pocket 25, with respective portions of the scalpel handle 19 disposed between the positioning bosses 30 and the alignment bosses 31, until the proximal edge 18f of the scalpel blade 19 is just past the positioning bosses 30 on the base 14, as seen in FIG. 9. The attaching tip 19b and engaging boss 19a of the scalpel handle 19 are positioned above the deeper portion 29 of the blade pocket 25. Next, in a step 203, the cover 12 is slid to the closed position illustrated in FIG. 1. The bottom edges (as seen in FIGS. 9 and 10) of the free ends 60 and 64 of the first lever arm 58 and the second lever arm 62, respectively, will engage the upper surface (as seen in FIGS. 9 and 10) of the scalpel blade 18. The cover 12 is able to be moved to the closed position with the scalpel handle 19 disposed in the device 10, because the recesses 71 and 72 in the bottom (as seen in FIGS. 9, 10A, 10B, 10C, and 10D) of the first lever arm 58 and the second lever arm 62, respectively, permit the first lever arm 58 and the second lever arm 62 to straddle the attaching tip 19b of the surgical handle 19 without engaging or being blocked by the attaching tip 19b during the reciprocating movement of the cover 12 between open and closed positions. In a fourth step 204, the user presses the free end 60 of the first lever arm 58 inwardly against the scalpel blade 18. This bends the shank portion 18b of the scalpel blade 18, so that proximal end of the shank portion 18b is free of the engaging boss 19a. The first lever arm 58 is kept depressed to clamp the shank end 18b between the first lever arm 58 and the base 14, as illustrated in FIGS. 10A and 10B. The first lever arm 58 does not bear against, and is not prevented from moving by, the scalpel handle 19 because of the recess 71 formed in the bottom (as seen in FIGS. 9 and 10) of the first lever arm 58. In a fifth step 205, the user pulls the scalpel handle 19 and the device 10 relatively apart, withdrawing the scalpel handle 19 to disengage the engaging boss 19a from the scalpel blade 18. Thus, the scalpel blade 18 is dismounted from the scalpel handle 19, and retained in the blade pocket 25 of the apparatus 10. Normally, as the engaging boss 19a of the scalpel handle 19 is withdrawn from the elongate aperture 18c of the scalpel blade 18, the scalpel blade 18 will straighten out, and move so that the boss 28 on the arm 27 of the base 14 will engage the elongate aperture 18c. Next, in a sixth step 206, the user relaxes the pressure on the first lever arm 58. The scalpel blade 18 is now wholly retained within the device 10 and completely removed from the scalpel handle 19 so that, in a final step 207, the device 10 (with the used blade 18 therein) may be disposed of, such as by placing the device 10 into a conventional “sharps” container. Of course, if desired and where appropriate, the above procedure for mounting the used scalpel blade 18 on the scalpel handle 19 can be repeated (for example, where the scalpel blade 18 is not used for a medical procedure, but rather as a hobby knife where sterile conditions need not be observed).

It will be appreciated by those skilled in the art, that the device 10 of the present invention is a decided improvement over the prior art and constitutes an important and valuable contribution in the art. The operation different and superior to the prior art, consisting of a reciprocating movement of the cover 12 relative to the base 14 between open and closed positions, together with a squeezing of a lever arm, which squeezing motion naturally forms part of the normal grasping of the device 10 by the user (in one hand) when inserting or withdrawing the scalpel handle 19, grasped with the other hand). The device 10 further acts as both a protective container for the surgical blade 18, and as a disposable package or container for the used scalpel blade 18. It is further noted that device 10 is usable with widely available standard scalpel blades with a single aperture formed through the scalpel blades; the device requires no modification of these blades to mount in the device 10 (in contrast, for example, U.S. Pat. No. 5,662,221 to Abidin et al., which, in addition to the aperture receiving the scalpel handle, requires another hole to be formed in the scalpel blade to receive a upstanding pin formed on the base).

If the additional locking feature shown in FIGS. 16-21 is utilized with the device it will be necessary to add an additional step to the procedure for using the device shown in FIG. 13. It will be necessary to disengage the first 331 and second 335 locking devices to allow the cover to move with respect to the base as previously described. However, the remains of the operation of the device will be as described in FIG. 13. With respect to the procedure shown in FIG. 14, again an additional step will be necessary to add another step to engage the first 331 and second 335 locking devices to secure the cover in the closed position on the base. However, the remainder of the operation will be as detailed in FIGS. 13 and 14.

Summarizing some of the other advantages and features of the invention, these include:

That the device 10 is capable of repeatedly dispensing and loading a blade (or tool tip) onto a handle, removing the blade from the handle after use, and safely containing the used blade for disposal or re-use, without the user having to touch the blade.

The device 10 may be color coded for the style (type and size) of blade to be contained therein, and size of associated fitment upon which the blade is to be mounted.

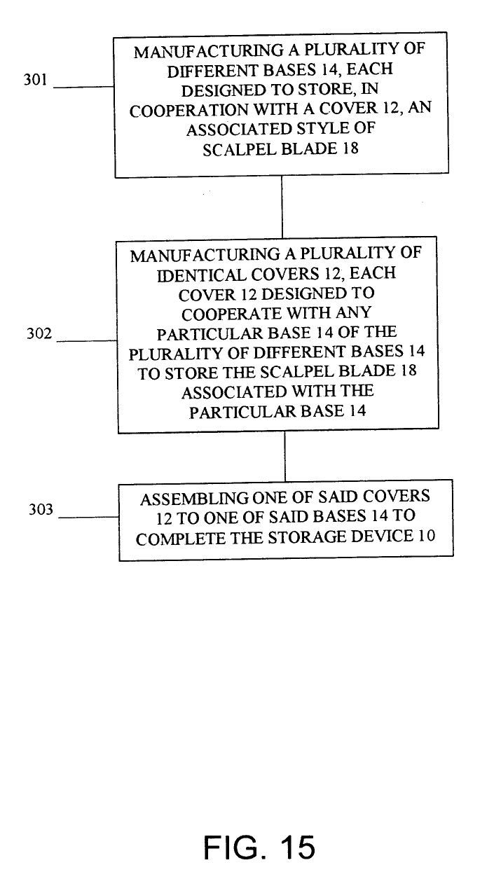

That the device is adaptable to a method of manufacturing the device for a variety of styles of scalpel blades, illustrated in FIG. 15, the method of manufacturing including:

in a first step 301, manufacturing a plurality of different bases 14, each designed to store, in cooperation with a cover, an associated style of scalpel blade 18;

in a second step 302, manufacturing a plurality of identical covers 12, each cover 12 designed to cooperate with any particular base 14 of the plurality of different bases 14 to store the scalpel blade 18 associated with the particular base 14; and

in a third step 303, assembling one of said covers 12 to one of said bases 14 to complete the storage device 10.

The scalpel blade 18 is contained in a blade pocket 25 sized to a particular type of scalpel blade and surrounding the sides and tip of the scalpel blade 18 to retain the scalpel blade 18 in position while stored, loading or unloading.

The device 10 is provided with stops in the form of the shoulders 40 and the reduced width portions 42 of the recesses 34 that prevent the cover 12 slidably mounted on the base 14 from being accidentally removed from the base 14.

The device 10 is provided with stops in the form of the reduced width portions 42 of the recesses 34 that retains the cover 12 partially over the scalpel blade 18 to assist in retaining the scalpel blade 18 in the device 10 when the cover 12 is open, until the scalpel blade 18 is deliberately removed by the user.

The device 10 can be used to both load and unload a scalpel blade 18 from a scalpel handle 19, and this can be done without directly handling the scalpel blade 18, or having the scalpel handle 19 unmounted from the scalpel handle 19 outside of the device 10, thus minimizing the chances of accidental cuts or pokes, and the chance of transmission of blood-borne diseases (AIDS, Hepatitis-B or other viral infections, etc.).

The device 10 may be provided with positioning bosses 30 and alignment bosses 31 to guide a scalpel handle 19 into the aperture 18c of a scalpel blade 18 stored in the device 10.

The device 10 may be provided with a body 33 that is radio-opaque.

The device 10 is provided with a body 33 that is magnetic.

The device is provided with a first and second locking device that releasably secure the cover in a closed position on the base. The first and second locking devices preventing accidental or unwanted removal of the blade from the blade pocket.

The above is a listing of just some of the objects and advantages of the invention; it is not complete, and should not be construed be the only advantages or objects of the invention, nor limit the scope of the invention. The principle and mode of operation of this invention have been explained and illustrated in a preferred embodiment. However, it must be understood that this invention may be practiced otherwise than as specifically explained and illustrated without departing from its spirit or scope.

The above detailed description of the present invention is given for explanatory purposes. It will be apparent to those skilled in the art that numerous changes and modifications can be made without departing from the scope of the invention. Accordingly, the whole of the foregoing description is to be construed in an illustrative and not a limitative sense, the scope of the invention being defined solely by the appended claims.

We claim:

1. A scalpel blade storage device, comprising:
a cover having a first lever arm and a second lever arm defined therein;
a base having a face, said face defining a blade pocket therein having a distal portion adapted to receive a blade portion of a surgical scalpel blade and a proximal portion adapted to receive a shank portion of a surgical scalpel blade, said base being disposed adjacent to said cover with said face defining said blade pocket facing said cover; and
a plurality of arms fixed to one of said base and said cover, said arms enveloping at least a portion of the other of said base and said cover to retain said base and said cover in a sliding relationship, said cover being movable between a first closed position in which said blade pocket is completely covered by said cover, and a second open position in which said blade pocket is at least partially uncovered.

2. A method of mounting a scalpel blade having an aperture onto a scalpel handle having an engaging boss, comprising:
a) obtaining a scalpel handle and a storage device having a scalpel blade disposed therein between a base and a cover of said storage device, said cover having a lever arm adapted to engage said scalpel blade when said scalpel blade is disposed between said base and said cover;
b) sliding said cover to an open position at least partially uncovering said scalpel blade;
c) pressing said lever arm against said scalpel blade to clamp said scalpel blade against said base;
d) inserting said engaging boss into said aperture of said scalpel blade;
e) relaxing the pressure on said lever arm to free said scalpel blade; and
f) withdrawing said scalpel blade, mounted on said scalpel handle, from said storage device.

3. The method of claim 2, further including a step c1, after step b) and before step d), of orienting a shoulder on said scalpel handle parallel to a proximal edge of said scalpel blade.

4. A method of mounting a tool tip having an aperture onto a tool handle having an engaging boss, comprising:
a) obtaining a tool handle and a storage device having a tool tip disposed therein between a base and a cover of said storage device, said cover having a lever arm adapted to engage said tool tip when said tool tip is disposed between said base and said cover;
b) sliding said cover to an open position at least partially uncovering said tool tip;
c) pressing said lever arm against said tool tip to clamp said tool tip against said base;
d) inserting said engaging boss into said aperture of said tool tip;
e) relaxing the pressure on said lever arm to free said tool tip; and
f) withdrawing said tool tip, mounted on said tool handle, from said storage device.

5. A method of unmounting a scalpel blade having an aperture from a scalpel handle having an engaging boss and storing said scalpel blade in a storage device having a base and a cover, said cover being slideably attached to said base and cooperating with said base to define a blade storage space, said cover having a lever arm adapted to engage said scalpel blade when said scalpel blade is disposed between said base and said cover in said storage space, comprising:
a) ensuring the cover 12 is in an open position in which said storage space is at least partially uncovered;
b) inserting the scalpel blade mounted on the scalpel handle into the blade storage space;
c) moving said cover to a closed position in which said blade storage space is covered;
d) pressing said lever arm against said scalpel blade to bend a portion of said scalpel blade free of said engaging boss; and
e) pulling said scalpel handle and said storage device relatively apart so as to withdraw said engaging boss out of said aperture in said scalpel blade, dismounting said scalpel blade from said scalpel handle and leaving said scalpel blade retained in said blade storage space of said storage device.

6. The method of claim 5, further including a step f) after step e) of releasing said lever arm.

7. The method of claim 6, further including a step g) after step e) of disposing of said storage device with said scalpel blade retained in said blade storage space.

8. The method of claim 5, further including a step c1, after step b) and before step d), of inserting said scalpel handle until a proximal edge of said scalpel blade is just past a pair of positioning bosses formed on said base.

9. A method of unmounting a tool tip having an aperture from a handle having an engaging boss and storing said tool tip in a storage device having a base and a cover, said cover being slideably attached to said base and cooperating with said base to define a tool tip storage space, said cover having a lever arm adapted to engage said tool tip when said tool tip is disposed between said base and said cover in said storage space, comprising:
a) ensuring the cover 12 is in an open position in which said storage space is at least partially uncovered;
b) inserting the tool tip mounted on the handle into the tool tip storage space;
c) moving said cover to a closed position in which said tool tip storage space is covered;
d) pressing said lever arm against said tool tip to bend a portion of said tool tip free of said engaging boss; and
e) pulling said handle and said storage device relatively apart so as to withdraw said engaging boss out of said aperture in said tool tip, dismounting said tool tip from said handle and leaving said tool tip retained in said tool tip storage space of said storage device.

10. A device comprising a base and a cover slidably mounted on the base, said base and said cover cooperating so as to enable repeatedly dispensing and loading a blade onto a handle, removing the blade from the handle after use, and safely containing the used blade for disposal or re-use without the user touching the blade.

11. A device for storing a scalpel blade comprising a base and a cover cooperating to contain the scalpel blade, at least one of said base and said cover being color-coded to provide an indication of the style of blade to be contained therein.

12. A method of manufacturing a storage device for a variety of styles of scalpel blades, comprising:
manufacturing a plurality of different bases, each designed to store, in cooperation with a cover, an associated style of scalpel blade;
manufacturing a plurality of identical covers, each designed to cooperate with any particular base of the plurality of different bases to store the scalpel blade associated with the particular base; and
assembling one of said covers to one of said bases to complete the storage device.

13. A storage and dispensing device for a scalpel blade comprising a plastic base and a plastic cover cooperating so as to be adapted to contain a scalpel blade, the device further comprising a body that is magnetic.

14. The storage and dispensing device of claim 13 wherein said body is radio-opaque.

15. The device of claim 1 wherein a first locking device is positioned on a portion of said cover that is adjacent said base, a second locking device positioned on said base for engaging said first locking device when said cover is in said first closed position whereby said first and second locking devices secure said cover in said first closed position.

16. The device of claim 15 wherein a portion of said cover is rotatably positioned with respect to said base whereby said cover can be rotated to move said first locking device from engagement with said second locking device whereby said cover can be moved to a second open position.

17. The device of claim 16 wherein the first and second locking devices can be a projection or a cavity wherein the first and second locking devices can not be the same component.

18. The device of claim 17 wherein a channel is positioned in alignment with said cavity to provide space for said projection when the cover and base are moved from said first closed position to said and second open position.

19. The device of claim 18 wherein said channel has a ramp section adjacent to said cavity whereby said ramp acts to displace said projection in a gradual manner to allow said projection to be positioned for engagement with said cavity.

20. The device of claim 19 wherein said ramp has a slope of about 20° to about 45°.

21. The device of claim 16 wherein said cover defines a pivot point that facilitates rotation of said cover around said pivot point to disengage said first locking device from said second locking device.

22. The device of claim 2 wherein a portion of said cover that extends from said pivot point has a reduced thickness to provide more clearance between said cover and said base to facilitate rotation of said cover to disengage said first locking device.

23. The device of claim 22 wherein at least one slot is provided in said portion of said cover that has reduced thickness to further facilitate rotation of said cover.

24. The method of claim 2 in which a first locking device on said base and a second locking device on said cover are disengaged to allow said cover to move to uncover said scalpel blade.

25. The method of claim 4 in which a first locking device on said base and a second locking device on said cover are disengaged to allow said cover to move to uncover said tool tip.

26. The method of claim 5 in which a first locking device on said base and a second locking device on said cover are disengaged to allow said cover to move to at least partially uncover said storage space.

27. The method of claim 9 in which a first locking device on said base and a second locking device on said cover are disengaged to allow said cover to move to at least partially uncover said storage space.

28. The device of claim 1 wherein a boss is positioned in said blade pocket, said boss disposed to engage an aperture in said scalpel blade to position said scalpel blade in said blade pocket.

29. The device of claim 1 wherein said blade pocket has a deeper portion that is disposed to receive a portion of a scalpel handle to allow said scalpel blade to be mounted on said scalpel handle.

30. The device of claim 1 wherein at least one positioning boss is positioned on said base adjacent said blade pocket, said at least one boss being disposed to engage a portion of a scalpel handle to position said scalpel handle for engaging said scalpel blade in said blade pocket.

31. The device of claim 30 wherein said scalpel blade has a proximal edge that is inclined to the longitudinal axis of said scalpel blade, said proximal edge of said scalpel blade being disposed to engage said scalpel handle to orient said scalpel handle to said scalpel blade to allow said scalpel handle to attach to said scalpel blade.

32. The device of claim 1 wherein a magnet is secured to said base, said magnet allowing said device to be removably magnetically secured to a metallic surface.

33. The device of claim 1 wherein a radio opaque material is secured to said base.

34. The device of claim 1 wherein a shoulder is formed on said base, said shoulder being disposed to engage said cover to prevent said cover from advancing past said second open position.

35. The device of claim 1 wherein identifying indicia is secured to said device to indicate the size of the scalpel blade contained in said blade pocket.

36. The device of claim 35 wherein said identifying indicia is a colored area wherein the color utilized identifies the size of said scalpel blade.
